(19) United States
(12) Patent Application Publication
(10) Pub. No.: US 2024/0276933 A1
(43) Pub. Date: Aug. 22, 2024

(54) USE OF HIGHER FATTY ALCOHOL IN PROMOTING LIGNIN SYNTHESIS AND INCREASING YIELD IN RICE AND WHEAT

(71) Applicant: Zhuhai Runnong Science and Technology Co. Ltd, Zhuhai (CN)

(72) Inventors: Xiaoming Peng, Zhuhai (CN); Yunhao Sun, Zhuhai (CN); Dianxian Cai, Zhuhai (CN); Guohui Yu, Zhuhai (CN); Di Qin, Zhuhai (CN)

(21) Appl. No.: 18/651,713

(22) Filed: May 1, 2024

Related U.S. Application Data
(63) Continuation of application No. PCT/CN2021/128781, filed on Nov. 4, 2021.

(51) Int. Cl. A01H 1/06; A01H 1/00

(52) U.S. Cl. CPC A01H 1/06; A01H 1/108

(57) ABSTRACT

The application of the aqueous emulsion containing higher fatty alcohol of the present disclosure on rice and wheat can significantly affect the transcription levels of genes related to the phenylpropanoid metabolic pathways in rice and wheat, thereby increasing the lignin content of rice and wheat, and increasing the lodging resistance of rice and wheat. In practical agricultural production, the application of the aqueous emulsion containing higher fatty alcohol of the present disclosure can improve the rust resistance, insect resistance, lodging resistance and storage ability of rice and wheat, thereby improving the quality and yield of rice and wheat.

### CROSS-REFERENCE TO RELATED APPLICATIONS

The present application is a Continuation Application of PCT Application No. PCT/CN2021/128781, filed on Nov. 4, 2021, the entire contents of which are hereby incorporated by reference.

### TECHNICAL FIELD

The present disclosure relates to the use of a higher fatty alcohol in promoting the lignin synthesis and increasing yield in rice and wheat.

### DESCRIPTION OF THE PRIOR ART

Rice and wheat are important sources of food for humans and important food crops in China. China is a big country of rice and wheat production and marketing. In 2020, the total rice output was 211.85 billion kg, and the total wheat output was 134.25 billion kg. The yield of these two food crops is related to the food security of China.

Diseases, insect pests and adverse lodging in rice and wheat cultivation lead to serious yield and quality decline. In production, some problems can be solved by spraying chemical pesticides or human adjustments, which, however, result in increased economic costs or environmental costs. Further, there are some other adversities, such as continuous rain and strong winds, that cannot be avoided through human intervention.

### SUMMARY OF THE DISCLOSURE

In order to overcome the shortcomings of the prior art, the object of the present disclosure is to provide a use of a higher fatty alcohol in promoting the lignin synthesis and increasing the yield in rice and wheat.

In order to solve the above problems, the present disclosure adopts the technical solution as follows:

The present disclosure provides a use of a higher fatty alcohol in preparing a preparation for increasing a transcription level of genes related to a phenylpropanoid metabolic pathway in rice, wherein the higher fatty alcohol is used to promote lignin synthesis in rice by increasing the transcription level of genes related to the phenylpropanoid metabolic pathway in rice. Furthermore, the higher fatty alcohol is used to improve lodging resistance of the rice by promoting the lignin synthesis in rice.

In another aspect, the present disclosure provides a use of a higher fatty alcohol in preparing a preparation for increasing a transcription level of genes related to a phenylpropanoid metabolic pathway in rice, wherein the higher fatty alcohol is used to improve storage stability of the rice by increasing the transcription level of genes related to the phenylpropanoid metabolic pathway in rice.

In another aspect, the present disclosure provides a use of a higher fatty alcohol in preparing a preparation for increasing a transcription level of genes related to a phenylpropanoid metabolic pathway in rice, wherein the higher fatty alcohol is used to increase rice yield by increasing the transcription level of genes related to the phenylpropanoid metabolic pathway in rice.

In another aspect, the present disclosure provides a use of a higher fatty alcohol in promoting lignin synthesis in wheat by increasing the transcription level of genes related to the phenylpropanoid metabolic pathway in wheat. Furthermore, the higher fatty alcohol is used to improve lodging resistance of the wheat by promoting the lignin synthesis in wheat.

In another aspect, the present disclosure provides a use of a higher fatty alcohol in preparing a preparation for increasing a transcription level of genes related to a phenylpropanoid metabolic pathway in wheat, wherein the higher fatty alcohol is used to increase wheat yield by increasing the transcription level of genes related to the phenylpropanoid metabolic pathway in wheat.

In the present disclosure, the higher fatty alcohol is one of dodecyl alcohol and cetyl alcohol or a mixture of dodecyl alcohol and cetyl alcohol.

Further, the preparation is an aqueous emulsion, which includes higher fatty alcohol, emulsifier, thickener, and water.

The preparation containing the higher fatty alcohol of the present disclosure is not limited to an aqueous emulsion. All dosage forms based on the inventive concept of the present disclosure are possible, such as wettable powders, emulsions, sprayable solutions, concentrated emulsions, aerosols, and seed coatings.

Compared with the prior art, the benefits of the present disclosure are as follows.

The application of the aqueous emulsion containing higher fatty alcohol of the present disclosure on rice and wheat can significantly affect the transcription levels of genes related to the phenylpropanoid metabolic pathways in rice and wheat, thereby increasing the lignin content of rice and wheat, and increasing the lodging resistance of rice and wheat. In practical agricultural production, the application of the aqueous emulsion containing higher fatty alcohol of the present disclosure can improve the rust resistance, insect resistance, and lodging resistance of rice and wheat, thereby improving the quality, yield, and storage stability of rice and wheat.

### DESCRIPTION OF EMBODIMENTS

### Example 1

This example provides an aqueous emulsion containing higher fatty alcohol, which is composed of the following components in weight percentage: 24% dodecyl alcohol, 3% cetyl alcohol, 3% emulsifier, 5% thickener, and the remaining is water.

In this example, the emulsifier is polyoxyethylene fatty acid, and the thickener is methyl cellulose.

This example also provides a preparation method for the above-mentioned aqueous emulsion, which includes the following steps:

placing dodecyl alcohol and cetyl alcohol into a container and heating to 60° C. to melt, adding emulsifier to the container, processing with a high-shear homogenizer at a rotational speed of 5000 rpm for 10 minutes, adding water at 60° C. to the container, processing with a high-shear homogenizer at a rotational speed of 10,000 rpm for 10 minutes, cooling to 40° C., adding thickener to the container and processing with a high-shear homogenizer at a rotational speed of 10,000 rpm for 30 minutes, thereby obtaining the aqueous emulsion.

### Example 2

This example provides an aqueous emulsion containing higher fatty alcohol, which is composed of the following components in weight percentage: 24% dodecyl alcohol, 3% emulsifier, 5% thickener, and the remaining is water.

In this example, the emulsifier is polyoxyethylene fatty acid, and the thickener is methyl cellulose.

This example also provides a preparation method for the above-mentioned aqueous emulsion, which includes the following steps:
placing dodecyl alcohol into a container and heating to 60° C. to melt, adding emulsifier to the container, processing with a high-shear homogenizer at a rotational speed of 5000 rpm for 10 minutes, adding water at 60° C. to the container, processing with a high-shear homogenizer at a rotational speed of 10,000 rpm for 10 minutes, cooling to 40° C., adding thickener to the container and processing with a high-shear homogenizer at a rotational speed of 10,000 rpm for 30 minutes, thereby obtaining the aqueous emulsion.

### Example 3

This example provides an aqueous emulsion containing higher fatty alcohol, which is composed of the following components in weight percentage: 3% cetyl alcohol, 3% emulsifier, 5% thickener, and the remaining is water.

In this example, the emulsifier is polyoxyethylene fatty acid, and the thickener is methyl cellulose.

This example also provides a preparation method for the above-mentioned aqueous emulsion, which includes the following steps:
placing cetyl alcohol into a container and heating to 60° C. to melt, adding emulsifier to the container, processing with a high-shear homogenizer at a rotational speed of 5000 rpm for 10 minutes, adding water at 60° C. to the container, processing with a high-shear homogenizer at a rotational speed of 10,000 rpm for 10 minutes, cooling to 40° C., adding thickener to the container and processing with a high-shear homogenizer at a rotational speed of 10,000 rpm for 30 minutes, thereby obtaining the aqueous emulsion.

### Example 4: Effect of Aqueous Emulsion Containing Higher Fatty Alcohol on Transcription Level of Genes Related to Phenylpropanoid Metabolic Pathway in Rice

The phenylpropanoid metabolic pathway is involved in the biosynthesis of phytoalexin and lignin. Phytoalexin among other physiological metabolites has a good defensive effect against pathogenic fungi such as serious rusts and pest infestations. Lignin is the main source for providing mechanical strength for stems among other parts. Increasing lignin has been proven to significantly improve the lodging resistance of crops. Up-regulation in this type of synthetic pathway can increase rice's resistance to rust, insects, and lodging. Additionally, increasing lignin has been shown to significantly increase seed storage time. The upregulation in this type of synthetic pathway can increase the storage stability of rice and wheat seeds.

The rice samples were treated with the aqueous emulsions containing higher fatty alcohol (with different higher fatty alcohol ratios) of the present disclosure, transcriptome sequencing was performed, and the effect of the aqueous emulsions containing higher fatty alcohol on the transcription level of genes related to the phenylpropanoid metabolic pathway in rice was analyzed.

Fatty alcohol sample: aqueous emulsions containing higher fatty alcohol of the present disclosure listed in Table 1 (with different higher fatty alcohol ratios)

TABLE 1
Sample number

| Sample | Sample | Sample | Sample | Sample |
|---|---|---|---|---|
| Number | S component | A Component | B Component | control |
| raw material | Example 1 | Example 2 | Example 3 | water |

Tested plant: rice (HuangHuaNian)

Culture conditions: 90/0 μmol m⁻² s⁻¹ (light intensity of light/dark cycle), 14 hours/10 hours (time of light/dark cycle), 28° C./25° C., 70% relative humidity, four weeks.

### (3) Sample Processing and Data Collection:

Healthy plants with similar growth status from groups were selected, the leaves of the plants were sprayed with a dilution of the above sample (900 times diluted with water) until the leaf surface is completely covered with the liquid film. The control group was sprayed with the same amount of sterile water for diluting the primary solution. A second spraying treatment was performed after 48 hours. Each group includes 3 biological replicates.

After 72 hours for the first treatment, 4 grams of leaf samples from each group were collected. After flash freezing in liquid nitrogen for 3 minutes, the samples were sent to the laboratory of Wuhan Metware Biotechnology Co., Ltd. in dry ice for transcriptome sequencing (all transcriptome sequencing data below are provided by Wuhan Metware Biotechnology Co., Ltd.).

### (4) Effect of Treatment on the Transcription Level of Genes Related to the Phenylpropanoid Metabolic Pathway

The effect of different treatments on the transcription level of genes related to the phenylpropanoid metabolic pathway in rice leaves are shown in Table 2. The results of the transcriptome sequencing show that treatment with dodecyl alcohol, cetyl alcohol, or a mixture of the two can all up-regulate the expression levels of some important genes. In general, the treatment with the mixture of dodecyl alcohol and cetyl alcohol is better than the treatment with dodecyl alcohol or cetyl alcohol in regulating gene transcription levels.

TABLE 2
difference ratio of transcription levels of genes related to
the phenylpropanoid metabolic pathway in the leaves of rice
plants in different treatment group (up-regulation fold)

| | Rice A/ | Rice B/ | Rice S/ |
|---|---|---|---|
| Name of protein encoded by genes | Control | Control | Control |
| shikimate O-hydroxycinnamoyltransferase | 4.69 | 2.29 | 4.48 |
| caffeoyl-CoA O-methyltransferase | 2.79 | 2.34 | 3.07 |
| Peroxidase | 5.93 | 3.17 | 6.44 |
| phenylalanine ammonia-lyase | 2.56 | 8.98 | 4.25 |
| beta-glucosidase | 3.94 | <2.00 | 8.05 |
| flavonol synthase | 3.09 | <2.00 | 4.02 |
| flavonoid 3′-monooxygenase | 2.64 | 3.14 | 5.92 |
| phenolic glucoside malonyltransferase 2-like | 4.69 | 3.528 | 3.75 |

### Example 5: Effect of Aqueous Emulsion Containing Higher Fatty Alcohol on the Transcription Level of Genes Related to the Phenylpropanoid Metabolic Pathway in Wheat

The phenylpropanoid metabolic pathway is involved in the biosynthesis of phytoalexin and lignin. Phytoalexin among other physiological metabolites has a good defensive effect against pathogenic fungi such as serious rusts and pest infestations. Lignin is the main source for providing mechanical strength for stems among other parts. Increasing lignin has been proven to significantly improve the lodging resistance of crops. Up-regulation in this type of synthetic pathway can increase rice's resistance to rust, insects, and lodging. Additionally, increasing lignin has been shown to significantly increase seed storage time. The upregulation in this type of synthetic pathway can increase the storage stability of rice and wheat seeds.

The wheat samples were treated with the aqueous emulsions containing higher fatty alcohol (with different higher fatty alcohol ratios) of the present disclosure, transcriptome sequencing was performed, and the effect of the aqueous emulsions containing higher fatty alcohol on the transcription level of genes related to the anabolism of the phenylpropanoid metabolic pathway in wheat was analyzed.
(1) Fatty alcohol sample: the same as Example 4
(2) Tested plant: wheat (Yunong 211)

Culture conditions: 80/0 μmol m⁻² s⁻¹ (light intensity of light/dark cycle), 14 hours/10 hours (time of light/dark cycle), 28° C./25° C., 70% relative humidity, four weeks.
(3) Sample processing and data collection: the same as Example 5, transcriptome sequencing was completed by Wuhan Metware Biotechnology Co., Ltd.
(4) Effect of treatment on the transcription level of genes related to the phenylpropanoid metabolic pathway

The effect of different treatments on the transcription level of genes related to the phenylpropanoid metabolic pathway in wheat leaves are shown in Table 3. The results of the transcriptome sequencing show that treatment with dodecyl alcohol, cetyl alcohol, or a mixture of the two can all up-regulate the expression levels of some important genes. In general, the treatment with the mixture of dodecyl alcohol and cetyl alcohol is better than the treatment with dodecyl alcohol or cetyl alcohol in regulating gene transcription levels.

TABLE 3
difference ratio of transcription levels of genes related to
the phenylpropanoid metabolic pathway in the leaves of wheat
plants in different treatment group (up-regulation fold)

| Name of protein | Wheat A/ | Wheat B/ | Wheat S/ |
|---|---|---|---|
| encoded by genes | Control | Control | Control |
| shikimate | 2.15 | 2.09 | 8.16 |
| O-hydroxycinnamoyltransferase | | | |
| caffeoyl-CoA O-methyltransferase | <2.00 | 2.06 | 2.06 |
| peroxidase | 14.17 | 2.07 | 88.03 |
| phenylalanine ammonia-lyase | 2.02 | 2.31 | 2.86 |
| beta-glucosidase | 2.64 | <2.00 | 4.77 |
| flavonol synthase | 2.08 | <2.00 | 6.86 |
| flavonoid 3′-monooxygenase | 2.14 | 3.41 | 4.72 |
| phenolic glucoside | 3.32 | 2.13 | 2.31 |
| malonyltransferase 2-like | | | |

### Example 6: Application Effect of Aqueous Emulsion Containing Higher Fatty Alcohol in Rice Field Production

Sampling: sample S is the aqueous emulsion containing higher fatty alcohol prepared according to Example 1 as the test group

Crop: rice Meixiangnian

Location: Mashi Town, Shixing County, Shaoguan City, Guangdong

Province

Method: a 3.33 ha test group and a 3.33 ha control group at the production base were selected for production according to Table 4.

TABLE 4
protocol for the aqueous emulsion containing higher
fatty alcohol of the present disclosure on rice

| Serial | | | |
|---|---|---|---|
| Number | Treatment Period | Test group | Control Group |
| 1 | tillering stage | diluting sample S 900 times with water | spraying with the same |
| | | and spraying once; | amount of water while |
| | | sample dosage: 30 mL/0.067 ha/time | treating the test group |
| 2 | heading stage | diluting sample S 900 times with water | spraying with the same |
| | | and spraying once; | amount of water while |
| | | sample dosage: 30 mL/0.067 ha/time | treating the test group |
| 3 | milk-ripe stage | diluting sample S 900 times with water | spraying with the same |
| | | and spraying once; | amount of water while |
| | | sample dosage: 30 mL/0.067 ha/time | treating the test group |

### Comparison of Application Effects

The rice in the test group treated with the aqueous emulsion containing higher fatty alcohol of the present disclosure had no damage from diseases and insect pests during the entire growth process, and had obvious advantages in growth and appearance, with greater plant height, darker and glossy functional leaf color, more effective panicles, more grains per panicle, and more compact panicles. However, the control group was significantly weaker in these aspects.

Rice rust was found in the rice in the control group, accounting for 11% of the total number of rice in the control group, but not in the test group. According to the investigation and statistical method of crop diseases, the disease index of rice rust in the control group was 0.352, and that in the test group was 0.0.

The application effect is as follows:

TABLE 5
test results of the aqueous emulsion containing higher
fatty alcohol of the present disclosure on rice

| Serial | | | |
|---|---|---|---|
| Number | Item | Test group | Control Group |
| 1 | average number of plants | 29.6 | 24.8 |
| | per panicle (plants) | | |
| 2 | average number of grains | 223.2 | 205.4 |
| | per plant (grains) | | |
| 3 | average plant height (cm) | 127.4 | 120.4 |
| 4 | yield estimate (grains) | 6606.72 | 5093.92 |
| 5 | rice rust disease index | 0.0 | 0.352 |

The rice in the test group treated with the aqueous emulsion containing higher fatty alcohol of the present disclosure was obviously taller than the plants in the control group and had more leaves. The flag leaves remained emerald green in the mature stage, and the functions were well preserved. The rice yield increase rate reached 22.9%. With respect to the disease, the test group had better resistance to rice rust and no rice rust was found, while 11% of the control group had varying degrees of rust.

### Example 7: Application Effect of Aqueous Emulsion Containing Higher Fatty Alcohol in Rice Field Production

Sampling: sample S is the aqueous emulsion containing higher fatty alcohol prepared according to Example 1 as the test group

Crop: rice Yinhuxiangnian

Location: Pingsha Town, Zhuhai City, Guangdong Province

Method: a 3.33 ha test group and a 3.33 ha control group at the production base were selected for production according to Table 4 of Example 7.

### Comparison of Application Effects

Compared with the control group, the rice in the test group treated with the aqueous emulsion containing higher fatty alcohol of the present disclosure had obvious advantages in terms of growth and appearance during the entire growth process, with greater plant height, longer root, thicker stem, darker and glossy functional leaf color.

During the test period, two typhoons were encountered in succession. No lodging was found in the rice in the test group, while the rice in the control group suffered from serious lodging, accounting for 25% of the lodging area. Rice borers and rice leaf rollers were found in the rice in the control group, but not in the rice in the test group.

The application effect is as follows:

TABLE 6
test results of the aqueous emulsion containing higher
fatty alcohol of the present disclosure on rice

| Serial | | Test | Control |
|---|---|---|---|
| Number | Item | group | Group |
| 1 | pests and diseases | Not found | Rice borer and |
| | | | rice leaf roller |
| | | | were found |
| 2 | anti-lodging performance | No lodging | Lodging occurred |
| | in typhoon | occurred | in 25% of the area |
| 3 | average plant height (cm) | 124.0 | 115.7 |
| 4 | number of tillers per plant | 39.1 | 21.2 |
| 5 | average stem diameter | 7.1 | 5.5 |
| | (mm) | | |
| 6 | average yield per plant (g) | 110.8 | 89.5 |
| 7 | setting rate | 0.926 | 0.863 |
| 8 | weight of 1,000 grains (g) | 25.133 | 24.689 |
| 9 | yield of wet grains | 653.9 | 518.5 |
| | (kg/0.067 ha) | | |

The rice in the test group treated with the aqueous emulsion containing higher fatty alcohol of the present disclosure had significantly greater plant height, longer root, thicker stem, darker and glossy functional leaf color than the control group, and the rice yield increase rate reached 26.1%. Especially, affected by two typhoons, 25% of the area in the control group was suffered from lodging, while the test group was unaffected. With respect to the disease, no obvious pests were found in the test group, while varying degrees of damage from rice borers and rice leaf rollers occurred in the control group.

### Example 8: Application Effect of Aqueous Emulsion Containing Higher Fatty Alcohol in Wheat Field Production

Sampling: sample S is the aqueous emulsion containing higher fatty alcohol prepared according to Example 1 as the test group

Crop: wheat Yunong 211

Location: Banmu Township, Qi County, Kaifeng City, Henan Province

Method: a 0.67 ha test group and a 0.67 ha control group at the production base were selected for production according to Table 7.

TABLE 7
protocol for the aqueous emulsion containing higher
fatty alcohol of the present disclosure on wheat

| Serial | | | |
|---|---|---|---|
| Number | Treatment Period | Test group | Control Group |
| 1 | after emergence | diluting sample S 900 times with water | spraying with the same |
| | | and spraying once; | amount of water while |
| | | sample dosage: 30 mL/0.067 ha/time | treating the test group |
| 2 | tillering stage | diluting sample S 900 times with water | spraying with the same |
| | | and spraying once; | amount of water while |
| | | sample dosage: 30 mL/0.067 ha/time | treating the test group |
| 3 | heading stage | diluting sample S 900 times with water | spraying with the same |
| | | and spraying once; | amount of water while |
| | | sample dosage: 30 mL/0.067 ha/time | treating the test group |
| 4 | milk-ripe stage | diluting sample S 900 times with water | spraying with the same |
| | | and spraying once; | amount of water while |
| | | sample dosage: 30 mL/0.067 ha/time | treating the test group |

### Comparison of Application Effects

Compared with the control group, the wheat in the test group treated with the aqueous emulsion containing higher fatty alcohol of the present disclosure had obvious advantages in terms of growth and appearance during the entire growth process, with greater plant height, longer root, thicker stem, darker and glossy leaf color, more effective panicles, and more grains per panicle.

During the test period, strong winds were encountered one after another. No lodging was found in the wheat in the test group, while lodging occurred in the wheat in the control group, accounting for 10% of the total area. Wheat rust and aphids were found in the wheat in the control group, accounting for 17% of the total wheat in the control group, but not in the wheat in the test group.

### Example 9: Storage Stability of Rice Applied with Aqueous Emulsion Containing Higher Fatty Alcohol

TABLE 8
protocol for higher fatty alcohol mixture of the present disclosure on rice

| Serial | | | |
|---|---|---|---|
| Number | Treatment Period | Test group | Control Group |
| 1 | tillering stage | diluting sample S 900 times with water | spraying with the same |
| | | and spraying once; | amount of water while |
| | | sample dosage: 30 mL/0.067 ha/time | treating the test group |
| 2 | heading stage | diluting sample S 900 times with water | spraying with the same |
| | | and spraying once; | amount of water while |
| | | sample dosage: 30 mL/0.067 ha/time | treating the test group |
| 3 | milk-ripe stage | diluting sample S 900 times with water | spraying with the same |
| | | and spraying once; | amount of water while |
| | | sample dosage: 30 mL/0.067 ha/time | treating the test group |

Sampling: sample S is the aqueous emulsion containing higher fatty alcohol prepared according to Example 1

Crop: rice Jingliangyou 1212

Location: Longping Hi-Tech Chunhua Base, Changsha City, Hunan Province

Method: a 0.33 ha test group and a 0.33 ha control group at the production base were selected for production according to Table 8

Hunan Central-South Grain and Oil Science Research Institute evaluated the rice yielded, and the results are shown in Table 9 (data and reports were provided by Hunan Central-South Grain and Oil Science Research Institute):

TABLE 9
evaluation of the rice yielded under the pilot of the higher
fatty alcohol mixture of the present disclosure on rice

| | | | 30-day artificial | |
|---|---|---|---|---|
| | Original | | aging experiment | |
| | test | control | test | control |
| Treatment condition | group | group | group | group |
| insect pest density | 0 | 0 | 31 | 71 |
| moisture | 10.30% | 10.10% | 11.00% | 11.00% |
| fatty acid value | 27.3 | 17.7 | 30.7 | 28.4 |
| bacterial load | 9400 | 16000 | 7000 | 6900 |
| brown rice rate | 73.80% | 74.10% | 73.50% | 73.30% |
| polished rice rate | 55.20% | 55.60% | 53.90% | 52.40% |
| yellow grain rice rate | 0% | 0% | 0% | 0.10% |
| tasting score | 81.5 | 73.1 | 76.1 | 74.6 |

As can be seen from Table 9, the test rice applied with the aqueous emulsion containing higher fatty alcohol of the present disclosure has high quality and good taste. The tasting score in the original or aged group is much higher than that in the control group, the insect pest density is small, with a good storage stability.

It can be concluded from Example 4 and Example 5 that using the aqueous emulsion containing higher fatty alcohol of the present disclosure and diluting it with water to treat rice and wheat can significantly affect the transcription levels of genes related to the phenylpropanoid metabolic pathways in rice and wheat. The field application effect in Example 6 and Example 7 shows that the aqueous emulsion containing higher fatty alcohol of the present disclosure can effectively improve the rust resistance, insect resistance and lodging resistance of rice, and improve the quality and yield. The field application effect in Example 8 shows that the aqueous emulsion containing higher fatty alcohol of the present disclosure can effectively improve the rust resistance, insect resistance and lodging resistance of wheat, improve quality and increase yield. The effect of the storage stability of rice in Example 9 shows that the test rice yielded by using the aqueous emulsion containing higher fatty alcohol of the present disclosure has high quality, good taste, and low density of protozoal pests, with a good storage stability.

It can be concluded from Examples 4 to 9 that the application of the aqueous emulsion containing higher fatty alcohol of the present disclosure on rice and wheat can significantly affect the transcription levels of genes related to the phenylpropanoid metabolic pathways in rice and wheat, thereby improving the rust resistance, insect resistance, lodging resistance and storage stability of rice and wheat. In practical agricultural production, the application of the aqueous emulsion containing higher fatty alcohol of the present disclosure can improve the rust resistance, insect resistance, and lodging resistance of rice and wheat, improve the quality of rice and wheat, increase production and improve storage stability.

The above embodiments are only preferred embodiments of the present disclosure and are not intended to limit the scope of protection of the present disclosure. Any non-substantive changes and substitutions made by those skilled in the art on the basis of the present disclosure fall within the claimed protection scope of the present disclosure.

1. Use of a higher fatty alcohol in preparing a preparation for increasing a transcription level of genes related to a phenylpropanoid metabolic pathway in rice or wheat, wherein the higher fatty alcohol is used to promote lignin synthesis in rice or wheat by increasing the transcription level of genes related to the phenylpropanoid metabolic pathway in rice or wheat.

2. The use of claim 1, wherein the higher fatty alcohol is used to improve lodging resistance of the rice or the wheat by promoting the lignin synthesis in rice or wheat.

3. The use of claim 1, wherein the higher fatty alcohol is one of dodecyl alcohol and cetyl alcohol or a mixture of dodecyl alcohol and cetyl alcohol.

4. The use of claim 3, wherein the preparation is an aqueous emulsion, which comprises higher fatty alcohol, emulsifier, thickener and water.

5. Use of a higher fatty alcohol in preparing a preparation for increasing a transcription level of genes related to a phenylpropanoid metabolic pathway in rice, wherein the higher fatty alcohol is used to improve storage stability of the rice by increasing the transcription level of genes related to the phenylpropanoid metabolic pathway in rice.

6. The use of claim 5, wherein the higher fatty alcohol is one of dodecyl alcohol and cetyl alcohol or a mixture of dodecyl alcohol and cetyl alcohol.

7. The use of claim 6, wherein the preparation is an aqueous emulsion, which comprises higher fatty alcohol, emulsifier, thickener and water.

8. Use of a higher fatty alcohol in preparing a preparation for increasing a transcription level of genes related to a phenylpropanoid metabolic pathway in rice or wheat, wherein the higher fatty alcohol is used to increase rice or wheat yield by increasing the transcription level of genes related to the phenylpropanoid metabolic pathway in rice or wheat.

9. The use of claim 8, wherein the higher fatty alcohol is one of dodecyl alcohol and cetyl alcohol or a mixture of dodecyl alcohol and cetyl alcohol.

10. The use of claim 9, wherein the preparation is an aqueous emulsion, which comprises higher fatty alcohol, emulsifier, thickener and water.
